(19) United States
(12) Patent Application Publication
(10) Pub. No.: US 2023/0241061 A1
(43) Pub. Date: Aug. 3, 2023

(54) METHODS AND PHARMACEUTICAL COMPOSITIONS FOR THE TREATMENT OF FGFR3-RELATED COGNITIVE DEFICIT

(71) Applicants: INSERM (INSTITUT NATIONAL DE LA SANTÉ ET DE LA RECHERCHE MÉDICALE), Paris (FR); ASSISTANCE PUBLIQUE-HÔPITAUX DE PARIS (APHP), Paris (FR); FONDATION IMAGINE, Paris (FR); UNIVERSITÉ PARIS CITÉ, Paris (FR); CENTRE NATIONAL DE LA RECHERCHE SCIENTIFIQUE (CNRS), Paris (FR)

(72) Inventors: Laurence LEGEAI-MALLET, Paris (FR); Franck OURY, PARIS (FR)

(21) Appl. No.: 18/001,420

(22) PCT Filed: Jun. 10, 2021
(86) PCT No.: PCT/EP2021/065696

§ 371 Date: Dec. 9, 2022

(30) Foreign Application Priority Data
Jun. 11, 2020 (EP) 20305641.1

(51) Int. Cl. A61K 31/506; A61P 25/28

(52) U.S. Cl. CPC A61K 31/506; A61P 25/28

(57) ABSTRACT

The present invention relates to methods and pharmaceutical compositions for the treatment of FGFR3 -related cognitive deficit. The inventors provide strong evidence that FGFR3 gain of function mutation expressed in the brain induces cognitive and behavior deficit independently of skull anomalies. To provide evidence that the constitutive activation of FGFR3 is responsible for these behavioral impairments, the inventors treated the Fgfr3 mice with a tyrosine kinase inhibitor using intraventricular injection of BGJ398 for seven days. The treatment rescues the anomalies in short-term learning and in coping strategy. The present invention relates to a method of treating a FGFR3 -related cognitive deficit in a subject suffering from FGFR3 -related skeletal disease in need thereof comprising administering to the subject a therapeutically effective amount of FGFR3 inhibitor.

### FIELD OF THE INVENTION

The present invention relates to methods and pharmaceutical compositions for the treatment of FGFR3-related cognitive deficit.

### BACKGROUND OF THE INVENTION

The Fibroblast Growth Factor Receptor (FGFR) family plays an important role in bone development and skeletal diseases. FGFR1, FGFR2 and FGFR3 missense mutations are responsible for a spectrum of syndromic craniosynostoses characterized by premature fusion of cranial sutures (Robin et al., 1993; Twigg and Wilkie, 2015). Two specific FGFR3 dominant mutations account for Crouzon syndrome with acanthosis nigricans (CAN [MIM 612247]), a rare syndromic craniosynostosis, and Muenke syndrome (MS [MIM 602849]), the most common syndromic craniosynostosis (Wilkie et al., 2010). CAN patients present with acanthosis nigricans but otherwise resemble to FGFR2-related Crouzon syndrome patients: they are characterized by the premature fusion of the coronal sutures of the skull, brachycephaly, midfacacial hypoplasia and cranio-vertebral junction anomalies (Arnaud-López et al., 2007; Di Rocco et al., 2011; Meyers et al., 1995; Mir A et al., 2013). CAN is defined by a single point mutation (p.Ala391Glu) localized in transmembrane domain of FGFR3 (Li et al., 2006; Meyers et al., 1995). The potential consequences of abnormal skull vault, skull base and facial growth include increased intracranial pressure, hearing and vision impairments, impaired brain blood flow, hindbrain malformation, syringomyelia, sleep apnea and multifactorial developmental delay. Several studies have reported cognitive deficit in FGFR3-related craniosynostoses characterized by impairment of memory capacity, attention, anxiety, and emotional control (Kruszka et al., 1993; Maliepaard et al., 2014; Yarnell et al., 2015). FGFR3 is widely recognized as an important regulator of the endochondral ossification. However, its role in sutural growth biology and membranous ossification are less known. The role of the p.Ala391Glu CAN mutation remains unexplored. The inventors generated the first CAN mouse model (Fgfr3^{A385E/+}) expressing a dominant p.Ala385Glu mutation. The Fgfr3^{A385E/+} mice showed an absence of craniosynostosis and normal craniocerebral proportion. In the central nervous system, FGFR3 is highly expressed in the hippocampus, brain structure essential for cognition mechanisms. The inventors hypothesized that p.Ala385Glu mutation could affect adult neurogenesis and cognitive capacity. To test this hypothesis and to define the impact of FGFR3 gain-of-function mutation in behavior, the inventors examined the neurogenesis in hippocampus and showed decreased proliferation associated with decreased size of granular layer in the dentate gyrus of Fgfr3^{A385E/+} mice. Moreover, Fgfr3^{A385E/+} mice showed hippocampal-dependent learning and memory impairments and abnormal coping strategy to an inescapable stress.

The inventors also studied the Hch mouse model Fgfr3^{N534Ks/+} expressing the most common HCH mutation, p.Asn540Lys localized in FGFR3 tyrosine kinase I domain. To test this hypothesis and to define the impact of FGFR3 gain-of-function mutation in behavior, the inventors performed a series of behavioral test on Fgfr3^{N534K/+} mice and its control littermates. As a result, they found that Fgfr3^{N534Ks/+} mice exhibited hippocampal-dependent memory impairments and abnormal coping strategy to an inescapable stress.

Here, the inventors provide strong evidence that FGFR3 gain of function mutation expressed in the brain induces cognitive and behavioral deficits independently of skull anomalies. To provide evidence that the constitutive activation of FGFR3 is responsible for these behavioral impairments, the inventors treated the Fgfr^{3A385E/+}mice and the Fgfr^{N53Ks/+} mice with a tyrosine kinase inhibitor using intraventricular injection of BGJ398 for seven days. The treatment rescues the anomalies in learning and memory, and in coping strategy. Thus, targeting FGFR3 offers a novel and efficient therapeutic perspective to treat cognitive disorders in chondrodysplasia and craniosynostoses.

### SUMMARY OF THE INVENTION

The present invention relates to methods and pharmaceutical compositions for the treatment of FGFR3-related cognitive deficit. In particular, the present invention is defined by the claims.

### DETAILED DESCRIPTION OF THE INVENTION

Fibroblast Growth Factor Receptor 3 (FGFR3) gain-of-function mutations (p.Ala391Glu) is responsible for a rare form of craniosynostosis: Crouzon syndrome with Acanthosis Nigricans (CAN). The patients with CAN are characterized by premature fusion of coronal sutures of the skull, midface hypoplasia, acanthosis nigricans and neurological impairment. FGFR3 is defined as a negative regulator of long bone growth. However, the role of the p.Ala391Glu CAN mutation in sutural growth biology of the skull remains unexplored. The inventors observed that the CAN mutation induced an overactivation of receptor independently of ligand and disturbed the protein maturation. The inventors generated the first CAN mouse model (Fgfr3^{A385E/+}) expressing a dominant p.Ala385Glu mutation and a HCH mouse model (Fgfr3^{Asn534Lys/+}) expressing a dominant p.Asn540Lys mutation. The Fgfr3^{A385E/+} mice showed an absence of craniosynostosis and normal craniocerebral proportion. However, analyzing adult hippocampus of these mice, the inventors showed that FGFR3 overactivation was associated to decrease dentate gyrus progenitor proliferation and neurogenesis. Consequently, behavioral tests were performed in Fgfr3^{A385E/+} mice and hippocampal-dependent memory impairment and abnormal coping strategy were observed. Lastly, using a specific FGFR3 inhibitor (BGJ398), the inventors inhibited the FGFR3 overactivation in the brain of Fgfr3^{A385E/+} mice thus restoring the behavioral and cognitive defects. This highlights for the first-time behavior anomalies associated to Fgfr3 overactivation in the brain. It allows a better understanding of the role played by FGFR3 in learning processes and emotional responses in craniosynostoses.

Therefore, the present invention relates to a method of treating a FGFR3-related cognitive deficit in a subject suffering from FGFR3-related skeletal disease in need thereof comprising administering to the subject a therapeutically effective amount of FGFR3 inhibitor.

The present invention also relates to a FGFR3 inhibitor for use in the treatment of FGFR3-related cognitive deficit in a subject suffering from FGFR3-related skeletal disease.

As used herein, the term “subject” denotes a mammal, such as a rodent, a feline, a canine, and a primate. Particularly, the subject according to the invention is a human. As used herein, the term “subject” encompasses “patient”.

In some embodiment, the subject of the present invention is suffering or will suffer from cognitive deficit.

In some embodiment, the subject of the present invention suffers or will suffer from a FGFR3 gain of function mutation expressed in the brain which induces cognitive and behavior deficit.

In some embodiment, the subject of the invention has or will have episodic memory deficits, antidepressant effect, anomalies in learning and stress response.

As used herein, the terms “FGFR3”, “FGFR3 tyrosine kinase receptor” and “FGFR3 receptor” are used interchangeably throughout the specification and refer to all of the naturally-occurring isoforms of FGFR3. An exemplary human amino acid sequence of FGFR3 is represented by SEQ ID NO:1.

SEQ ID NO:1 >sp|P22607|FGFR3_HUMAN Fibrobla
st growth factor receptor 3 OS=Homo sapiens
OX=9606 GN=FGFR3 PE=1 SV=1 MGAPACALALCVAVAIV
AGASSESLGTEQRVVGRAAEVPGPEPGQQEQLVFGSGDAVELS
CPPPGGGPMGPTVWVKDGTGLVPSERVLVGPQRLQVLNASHEDSGAYSCR
QRLTQRVLCH FSVRVTDAPSSGDDEDGEDEAEDTGVDTGAPYW
TRPERMDKKLLAVPAANTVRFRCPAAG NPTPSISWLKNGREFR
GEHRIGGIKLRHQQWSLVMESVVPSDRGNYTCVVENKFGSIRQT
YTLDVLERSPHRPILQAGLPANQTAVLGSDVEFHCKVYSDAQPHIQWLK
HVEVNGSKVGP DGTPYVTVLKTAGANTTDKELEVLSLHNVTFE
DAGEYTCLAGNSIGFSHHSAWLVVLPAE EELVEADEAGSVYAG
ILSYGVGFFLFILVVAAVTLCRLRSPPKKGLGSPTVHKISRFPLK
RQVSLESNASMSSNTPLVRIARLSSGEGPTLANVSELELPADPKWELS
RARLTLGKPLGE GCFGQVVMAEAIGIDKDRAAKPVTVAVKMLK
DDATDKDLSDLVSEMEMMKMIGKHKNIIN LLGACTQGGPLYVL
VEYAAKGNLREFLRARRPPGLDYSFDTCKPPEEQLTFKDLVSCAYQ
VARGMEYLASQKCIHRDLAARNVLVTEDNVMKIADFGLARDVHNLDY
YKKTTNGRLPVKW MAPEALFDRVYTHQSDVWSFGVLLWEIFTL
GGSPYPGIPVEELFKLLKEGHRMDKPANCT HDLYMIMRECWHA
APSQRPTFKQLVEDLDRVLTVTSTDEYLDLSAPFEQYSPGGQDTPSS
SSSGDDSVFAHDLLPPAPPSSGGSRT

As used herein, the expressions “constitutively active FGFR3 receptor variant”, “constitutively active mutant of the FGFR3” or “mutant FGFR3 displaying a constitutive activity” are used interchangeably and refer to a mutant of said receptor exhibiting a biological activity (i.e. triggering downstream signaling), and/or exhibiting a biological activity which is higher than the biological activity of the corresponding wild-type receptor in the presence of FGF ligand. A constitutively active FGFR3 variant according to the invention is in particular chosen from the group consisting of (residues are numbered according to their position in the precursor of fibroblast growth factor receptor 3 isoform 1 - 806 amino acids long -): a mutant wherein the serine residue at position 84 is substituted with lysine (named herein below S84L); a mutant wherein the arginine residue at position 200 is substituted with cysteine (named herein below R200C); a mutant wherein the arginine residue at position 248 is substituted with cysteine (named herein below R248C); a mutant wherein the serine residue at position 249 is substituted with cysteine (named herein below S249C); a mutant wherein the proline residue at position 250 is substituted with arginine (named herein below P250R); a mutant wherein the asparagine residue at position 262 is substituted with histidine (named herein below N262H); a mutant wherein the glycine residue at position 268 is substituted with cysteine (named herein below G268C); a mutant wherein the tyrosine residue at position 278 is substituted with cysteine (named herein below Y278C); a mutant wherein the serine residue at position 279 is substituted with cysteine (named herein below S279C); a mutant wherein the glycine residue at position 370 is substituted with cysteine (named herein below G370C); a mutant wherein the serine residue at position 371 is substituted with cysteine (named herein below S371C); a mutant wherein the tyrosine residue at position 373 is substituted with cysteine (named herein below Y373C); a mutant wherein the glycine residue at position 380 is substituted with arginine (named herein below G380R); a mutant wherein the valine residue at position 381 is substituted with glutamate (named herein below V381E); a mutant wherein the alanine residue at position 391 is substituted with glutamate (named herein below A391E); a mutant wherein the asparagine residue at position 540 is substituted with Lysine (named herein below N540K); a mutant wherein the termination codon is eliminated due to base substitutions, in particular the mutant wherein the termination codon is mutated in an arginine, cysteine, glycine, serine or tryptophane codon (named herein below X807R, X807C, X807G, X807S and X807W, respectively); a mutant wherein the lysine residue at position 650 is substituted with another residue, in particular with methionine, glutamate, asparagine or glutamine (named herein below K650M, K650E, K650N and K650Q); a mutant wherein the methionine residue at position 528 is substituted with isoleucine (named herein below M528I); a mutant wherein the isoleucine residue at position 538 is substituted with valine (named herein below I538V); a mutant wherein the asparagine residue at position 540 is substituted with serine (named herein below N540S); a mutant wherein the asparagine residue at position 540 is substituted with threonine (named herein below N540T). Typically, a constitutively active FGFR3 variant according to the invention is N540K, K650N, K650Q, M528I, I538V, N540S, N540T or A391E mutant.

As used herein, the term “cognitive deficit” relates to a set of symptoms including depression, memory, perception, slowness, and difficulty solving problems. Cognitive deficit may exist as symptoms in some mental disorders (psychoses, mood disorders, anxiety disorders), but they are primarily synonymous with brain damage.

As used herein, the term “FGFR3-related cognitive deficit” is intended to mean a cognitive deficit that is caused by an abnormal over-activation of FGFR3 in the brain, in particular by expression of a constitutively active mutant of the FGFR3receptor, in particular a constitutively active mutant of the FGFR3 receptor as described above.

In some embodiment, the subject having FGFR3-related cognitive deficit suffers from FGFR3-related skeletal disease.

As used herein, the term “neurogenesis” has its general meaning in the art and relates to the process by which new neurons are formed in the brain. Neurogenesis is crucial when an embryo is developing, but also continues in certain brain regions after birth and throughout our lifespan. The mature brain has many specialised areas of function, and neurons that differ in structure and connections. The hippocampus, for example, which is a brain region that plays an important role in memory and spatial navigation, alone has at least 27 different types of neurons. The incredible diversity of neurons in the brain results from regulated neurogenesis during embryonic development. During the process, neural stem cells differentiate-that is, they become any one of a number of specialised cell types-at specific times and regions in the brain.

As used herein the term “FGFR3-related skeletal disease” is intended to mean a skeletal disease that is caused by an abnormal increased activation of FGFR3, in particular by expression of a constitutively active mutant of the FGFR3 receptor, in particular a constitutively active mutant of the FGFR3 receptor as described above.

In some embodiment, the FGFR3-related skeletal diseases are preferably FGFR3-related chondrodysplasias and FGFR3-related craniosynostosis.

As used herein “FGFR3-related chondrodysplasias” include but are not limited to dwarfism such as hypochondroplasia (HCH), thanatophoric dysplasia (TD) type I, thanatophoric dysplasia type II, achondroplasia (ACH) and SADDAN (severe achondroplasia with developmental delay and acanthosis nigricans).

In particular, the FGFR3-related skeletal disease is dwarfism.

As used herein, the term “dwarfism” has its general meaning in the art and refers to a short stature that results from a genetic or medical condition. Dwarfism is generally defined as an adult height of 147 centimeters or less.

In particular, the FGFR3-related skeletal disease is hypochondroplasia (HCH).

As used herein, the term “hypochondroplasia” (HCH) has its general meaning in the art and relates to a disproportionately short stature, micromelia and a head that appears large in comparison with the underdeveloped portions of the body.

In some embodiment, the FGFR3-related chondrodysplasias is a hypochondroplasia caused by expression of the N540K, K650N, K650Q, M528I, I538V, N540S or N540T constitutively active mutant of the FGFR3 receptor.

In particular, the FGFR3-related skeletal disease is achondroplasia (ACH).

As used herein, the term “achondroplasia” (ACH) has its general meaning in the art and relates to a genetic deficit in which the arms and legs are short, while the torso is typically of normal length and with an enlarged head and prominent forehead.

In particular, the FGFR3-related skeletal disease is thanatophoric dysplasia (TD).

As used herein, the term “thanatophoric dysplasia” (TD) has its general meaning in the art and relates to a severe skeletal deficit characterized by a disproportionately small ribcage, extremely short limbs and folds of extra skin on the arms and legs.

In some embodiment, the FGFR3-related skeletal disease is FGFR3-related craniosynostosis. In some embodiments, the FGFR3-related craniosynostosis corresponds to an inherited or to a sporadic disease.

In particular, the FGFR3-related craniosynostosis is Muenke syndrome caused by expression of the P250R constitutively active mutant of the FGFR3 receptor.

In particular, the FGFR3-related craniosynostosis is Crouzon syndrome with acanthosis nigricans (CAN) caused by expression of the A391E constitutively active mutant of the FGFR3 receptor.

As used herein, the term “craniosynostosis” has its general meaning in the art and relates a condition in which one or more of the fibrous sutures a subject skull prematurely fuses by turning into bone (ossification), thereby changing the growth pattern of the skull. “Crouzon syndrome with acanthosis nigricans” (CAN) is a very rare craniosynostosis.

As used herein, the term “acanthosis nigricans” relates to a brown to black, poorly defined, velvety hyperpigmentation of the skin.

As used herein, the term “FGFR3^{Y367C/+}” relates to a mouse model that recapitulates the human ACH phenotype. The clinical hallmarks of ACH (e.g. dwarfism, associated with reduced size of the foramen magnum, hypoplasia of the mandibles, hearing loss, anomalies of the intervertebral discs (Pannier et al. 2009, 2010, Mugniery et al 2012, Di Rocco et al 2014, Komla Ebri et al 2016).

As used herein, the term “FGFR3^{N534K/+}” relates to a HCH mouse model. The mutant mice display the clinical features of HCH with growth defects, growth plate anomalies, partial loss of synchondrosis and lordosis.

As used herein, the term “FGFR3^{A385E/+}” relates to a CAN mouse model in which a defective memory was observed.

As used herein, the term “treatment” or “treat” refer to both prophylactic or preventive treatment as well as curative, improving the patient’s condition or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical deficit or who ultimately may acquire the deficit, in order to prevent, cure, delay the onset of, reduce the severity of, or ameliorate one or more symptoms of a deficit or recurring deficit, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., daily, weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “preventing” intends characterizing a prophylactic method or process that is aimed at delaying or preventing the onset of a deficit or condition to which such term applies.

The term “expression” when used in the context of expression of a gene or nucleic acid refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein (i.e. FGFR3) produced by translation of a mRNA.

As used herein, the term “inhibitor” as used herein includes not only drugs for inhibiting activity of target molecules, but also drugs for inhibiting the expression of target molecules.

The inhibitor according to the invention are capable of inhibiting or eliminating the functional activation of the FGFR3 receptor in vivo and/or in vitro. The inhibitor may inhibit the functional activation of the FGFR3 receptor by at least about 10%, preferably by 20 at least about 30%, preferably by at least about 50%, preferably by at least about 70, 75 or 80%, still preferably by 85, 90, 95, or 100%.

The inhibitors according to the present invention include those which specifically bind to the FGFR3 receptor, thereby reducing or blocking signal transduction. Antagonists of this type include antibodies (in particular the antibodies as disclosed above) or aptamers which bind to FGFR3, fusion polypeptides, peptides, small chemical molecules which bind to FGFR3, and peptidomimetics.

As used herein the term “polypeptide” refers to any chain of amino acids linked by peptide bonds, regardless of length or post-translational modification. Polypeptides include natural proteins, synthetic or recombinant polypeptides and peptides (i.e. polypeptides of less than 50 amino acids) as well as hybrid, post-translationally modified polypeptides, and peptidomimetic.

As used herein, the term “amino acid” refers to the 20 standard alpha-amino acids 10 as well as naturally occurring and synthetic derivatives. A polypeptide may contain L or D amino acids or a combination thereof.

As used herein the term “peptidomimetic” refers to peptide-like structures which have non-amino acid structures substituted but which mimic the chemical structure of a peptide.

The term “antibody” refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments.

In particular, the antibody according to the invention may correspond to a polyclonal antibody, a monoclonal antibody (e.g. a chimeric, humanized or human antibody), a fragment of a polyclonal or monoclonal antibody or a diabody. “Antibody fragments” comprise a portion of an intact antibody, preferably the antigen binding or variable region of the intact antibody. Examples of antibody fragments include Fv, Fab, F(ab′)2, Fab′, Fd, dAb, dsFv, scFv, sc(Fv)2, CDRs, diabodies and multispecific antibodies formed from antibodies fragments.

Antibodies according to the invention may be produced by any technique known in the art, such as, without limitation, any chemical, biological, genetic or enzymatic technique, either alone or in combination. The antibodies of this invention can be obtained by producing and culturing hybridomas.

“Aptamers” are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., Science, 1990, 249(4968):505-10. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S.D., Clin. Chem., 1999, 45(9):1628-50. Peptide aptamers consists of a conformationally constrained antibody variable region displayed by a platform protein, such as E. coli Thioredoxin A that are selected from combinatorial libraries by two hybrid methods (Colas et al., Nature, 1996,380, 548-50).

The term “small chemical molecule” refers to a molecule, preferably of less than 1,000 daltons, in particular organic or inorganic compounds. Structural design in chemistry should help to find such a molecule.

In some embodiments, the FGFR3 inhibitor is a tyrosine kinase inhibitor.

The present invention relates to a method of treating a FGFR3-related cognitive deficit in a subject suffering from FGFR3-related skeletal disease in need thereof comprising administering to the subject a therapeutically effective amount of tyrosine kinase inhibitor (TKI).

The present invention also relates to a tyrosine kinase inhibitor for use in the treatment or prevention of FGFR3-related cognitive deficit in a subject suffering FGFR3-related skeletal disease

As used herein, the term “tyrosine kinase inhibitor” (TKI) refers to a compound (natural or synthetic) which is effective to inhibit tyrosine kinase activity. In addition, the inhibitors with a specific activity on tyrosine kinase may be preferred.

Examples of tyrosine kinase inhibitor include but are not limited to PD173074 (CAS No. 219580-11-7), AZD4547 (CAS No. 1035270-39-3), BGJ398 (CAS No. 872511-34-7), AP24534 (CAS No. 943319-70-8), BIBF1120 (CAS No. 656247-17-5), JNJ-42756493 (CAS No. 1346242-81-6), TKI-258 (CAS No. 405169-16-6), PHA- 739358 (CAS No. 827318-97-8), BMS-540215 (CAS No. 649735-46-6), TKI-258 dilactic acid (CAS No. 852433-84-2), MK-2461 (CAS No. 917879-39-1), BMS-582664 (CAS No. 649735-63-7), SSR128129E (CAS No. 848318-25-2), PRN1371 (CAS No. 1802929-43-6), PD166866 (CAS No. 192705-79-6), BLU554 (CAS No. 1707289-21-1), S49076 (CAS No. 1265965-22-7), SU5402 (CAS No. 215543-92-3), BLU9931 (CAS No. 1538604-68-0), FIN-2 (CAS No. 1633044-56-0), TKI-258 lactate (CAS No. 915769-50-5), CH5183284 (CAS No. 1265229-25-1), LY2874455 (CAS No. 1254473-64-7) or ASP5878 (CAS No. 1453208-66-6). As one is well aware, the CAS (chemical abstracts service) number assigned to each molecule is a unique identifier for each compound.

In a particular embodiment, the tyrosine kinase inhibitor is BGJ398, a potent inhibitor of the FGFR family. As used herein, the term “BGJ398” has its general meaning in the art and refers to 3-(2,6-dichloro-3,5-dimethoxyphenyl)-1-[6-[4-(4-ethylpiperazin-1-yl)anilino]pyrimidin-4-yl]-1-methylurea. The term is also known as Infigratinib, NVP-BGJ398, or BGJ-398.

As used herein the terms “administering” or “administration” refer to the act of injecting or otherwise physically delivering a substance as it exists outside the body (e.g. a FGFR3 inhibitor) into the subject, such as by intracerebroventricular, mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

A “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the FGFR3 inhibitor are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. One of ordinary skill in the art would be able to determine such amounts based on such factors as the subject’s size, the severity of the subject’s symptoms, and the particular composition or route of administration selected. An exemplary, non-limiting range for a therapeutically effective amount of drug is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. Administration may e.g. be intra-cerebro-ventricular, intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. As non-limiting examples, treatment according to the present invention may be provided as a daily dosage of the agent of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

Accordingly, the subject is administered with a pharmaceutical composition comprising the FGFR3inhibitor as active principle and at least one pharmaceutically acceptable excipient. As used herein the term “active principle” or “active ingredient” are used interchangeably. The active principle is used to alleviate, treat or prevent a medical condition or disease. By the term “pharmaceutically acceptable excipient” herein, it is understood a carrier medium which does not interfere with the effectiveness of the biological activity of the active ingredient(s) and which is not excessively toxic to the host at the concentration at which it is administered. Said excipients are selected, depending on the pharmaceutical form and the desired method of administration, from the usual excipients known by a person skilled in the art. In some embodiments, the pharmaceutical composition of the present invention does not comprise a second active principle.

The present invention also provides for therapeutic applications where the FGFR3 inhibitor of the present invention is used in combination with at least one further therapeutic agent, e.g. for treating cognitive deficit. Such administration may be simultaneous, separate or sequential. For simultaneous administration the agents may be administered as one composition or as separate compositions, as appropriate. The further therapeutic agent is typically relevant for the deficit to be treated.

As used herein, the term “combination” is intended to refer to all forms of administration that provide a first drug together with a further (second, third...) drug. The drugs may be administered simultaneously, separately or sequentially and in any order. According to the invention, the drug is administered to the subject using any suitable method that enables the drug to reach the brain. In some embodiments, the drug administered to the subject systemically (i.e. via systemic administration). Thus, in some embodiments, the drug is administered to the subject such that it enters the circulatory system and is distributed throughout the body. In some embodiments, the drug is administered to the subject by local administration, for example by local administration to the hypothalamus.

As used herein, the terms “combined treatment”, “combined therapy” or “therapy combination” refer to a treatment that uses more than one medication. The combined therapy may be dual therapy or bi-therapy.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

The invention will be further illustrated by the following figures and examples. However, these examples and figures should not be interpreted in any way as limiting the scope of the present invention.

### FIGURES

FIG. 1: Learning impairment and antidepressant-like behavior in CAN model.

A. Novel object recognition (NOR) performed in 4 months old male animals. Discrimination index was measured 24 h after training phase to assess memory performance of Fgfr3^{A385E/+} and their control littermates. NOR was performed on two independent groups of Fgfr3^{A385E/+} (n=17) and their controls (n=14). B. Contextual Fear Conditioning (CFC) performed in 4 month-old male animals (Fgfr3^{+/+}, n=13; Fgfr3^{A385E/+} n=12). Percent freezing time was recorded for training phase (as control for basal level) and testing phase (to assess memory performance). C. Forced swim test (FST) performed in 4 month-old male animals in two independent groups. FST was performed during two consecutive days. Immobility duration was assessed for both day 1 and day 2. (Fgfr3^{+/+}, n=17; Fgfr3^{A385E/+}, n=14). D. Tail suspension test (TST) performed in 4 month-old male animals during two consecutive days and immobility duration was assessed for both. (Fgfr3^{+/+}, n=17; Fgfr3^{A385E/+} n=14). All behavioral analyses were performed on two independent cohorts of mice and two independent experiments for each group.

FIG. 2: Downregulation of Fgfr3^{A385E/+} phosphorylation in hippocampus restored memory deficit and antidepressant-like behavior.

A. Local daily intracerebroventricular injection with either BGJ398 or vehicle in 4 month-old male animals during 7 days. NOR training session occurred 1 hour after last injection B. NOR performed on groups injected with vehicle or BGJ398. Injection and NOR were performed on two independent groups with the three conditions each (Fgfr3^{+/+} + vehicle, n=15; Fgfr3^{A385E/+} + vehicle, n=14; Fgfr3^{A385E/+} + BGJ398, n=14. C. Local daily intracerebroventricular injection with either BGJ398 or vehicle in 4 month-old male animals during 7 days. One FST testing session occurring 1 hour after last injection. Immobility duration was assessed during testing. D. FST performed on groups injected with vehicle or BGJ398. Injection and FST were performed on two independent groups with the three conditions each (Fgfr3^{+/+} + vehicle, n=15; Fgfr3^{A385E/+} + vehicle, n=14; Fgfr3^{A385E/+} + BGJ398, n=14). *P<0.05 **P<0.01, ***P<0.001.NS Not significant by ANOVA with Tukey’s multiple comparisons post-test and Student’s unpaired t test.

FIG. 3: Learning impairment is reverse by FGFR3 tyrosine kinase inhibitor daily subcutaneous injection of BGJ398. A. Novel object location (NOL) was performed in 4 months old male animals. Discrimination index was measured 24 h after training phase to assess memory performance of Fgfr3Asn534Lys/+ and their control littermates. B. Novel object recognition (NOR) was performed in 4 months old male animals. Discrimination index was measured 24 h after training phase to assess memory performance of Fgfr3Asn534Lys/+ + BGJ398, Fgfr3Asn534Lys/+ and their control littermates. NOR and NOL were performed on two independent groups of Fgfr3Asn534Lys/+ (n=12) Fgfr3Asn534Lys/+ + BGJ398 (n=14) and their controls (n=14).

### MATERIAL AND METHODS

All procedures were approved by the French Animal Care and Use Committee. Genomic DNA was isolated from tail using NucleoSpin Tissue kit (Macherey-Nagel) according the manufacturer’s instructions. The mice were genotyped using the following primers: 5′-GTGGGGGTTCTGCGGTTGG-3′ (SEQ ID NO: 2) and 5′-TGACAGGCTTGGCAGTACGG-3′ (SEQ ID NO: 3) for isolate WT and mutant mice. For all analyses, wild-type littermates were used as controls.

### Brain MRI

Mice brain MRI were acquired at the Small Animal Imaging Platform (Faculté de Médecine, Université Paris Descartes Sorbonne Paris Cité, Paris, France) using a small-animal 4.7-T MR imaging unit (Biospec 47/40; Bruker, Billerica, MA, USA) with a resolution of 100 µm. Fgfr3^{A385E/+} mice and 5 Fgfr3^{Asn534Lys/+} male mice and 5 controls littermates of 4 months-of-age were anesthetized with isoflurane gas inhalation during acquisition. Three-dimensional reconstruction and measurement were performed using Imaris (Bitplane).

### Surgery and Drug Treatment

BGJ398 was purchased from LC Laboratories, Woburn, MA, USA. For BGJ398 injection, 4 months old Fgfr3^{Asn534Lys/+} mice and Fgfr3^{A385E/+} mice received daily sub-cutaneous administration of BGJ398 (2 mg/kg) or vehicle (HcL 3.5 mM, DMSO 2%) for six days. For habituation and testing NOR and NOL, injection was performed 1 hour before testing phase.

### Behavior Tests (Commons Et Al. 2017; Glatigny Et Al. 2019)

### 3-Foot Shock Contextual Fear Conditioning (CFC)

Mice were transported a short distance from the holding mouse facility to the testing room in their home cages and left undisturbed for at least one hour before the beginning of the test. The conditioning chambers were obtained from Bioseb (France) with internal dimensions of 25 × 25 × 25 cm. Each chamber was located inside a larger, insulated plastic cabinet that provided protection from outside light and noise (67 × 55 × 50 cm, Bioseb, France), and mice were tested individually in the conditioning boxes. Floors of the chamber consisted of 27 stainless steel bars wired to a shock generator with scrambler for the delivery of foot shock. Signal generated by the mice movements was recorded and analyzed through a high sensitivity weight transducer system. The analog signal was transmitted to the Freezing software module through the load cell unit for recording purposes and analysis of time active/time immobile (Freezing) was performed. The CFC procedure took place over two consecutive days. On day 1, mice were placed in the conditioning chamber, and received 3 foot-shocks (1.5 sec, 0.5 mA), which were administrated at 60, 120 and 180 sec after the animals were placed in the chamber. They were returned to their home cages, 60 sec after the final shock. Contextual fear memory was assessed 24 hours after conditioning by returning the mice to the conditioning chamber and measuring freezing behavior during a 4 min retention test. Freezing was scored and analyzed automatically using Packwin 2.0 software (Bioseb, France). Freezing behavior was considered to occur if the animals froze for a period of at least two seconds. Behavior was scored by the Freezing software.

### Novel Object Recognition Paradigm (NOR)

We used a modified version of the NOR task as described in Glatigny et al., 2019. Mice were transported a short distance from the holding mouse facility to the testing room in their home cages and left undisturbed for at least one hour before the beginning of the test. The testing room was lit with two 60-W light bulbs and behavior sessions were recorded with a camera above the testing arena (grey plastic box (60 × 40 × 32 cm)). Mice could not contact or see each other during the exposures. The light intensity was equal in all parts of the arena (approximately 20 lx). Two different objects were used, available in triplicate. The objects were a blue ceramic pot (diameter 6.5 cm, maximal height 7.5 cm) and a clear, plastic funnel (diameter 8.5 cm, maximal height 8.5 cm). The objects that serve as a novel object, as well as the left/right localization of the objects, were counterbalanced within each group. The objects elicited equal levels of exploration as determined in pilot experiments and training phase. Between exposures, mice were held individually in standard cages, the objects and arenas were cleaned with phagosphore, and the bedding replaced.

The NOR paradigm consists of three phases (over 3 days): a habituation phase, a training phase, and a testing phase. Mice were always placed in the center of the arena at the start of each exposure. On day 1: the habituation phase, mice were given 5 min to explore the arena, without any objects and were then taken back to their home cage. On day 2, the training phase, mice were allowed to explore, for 10 min, two identical objects arranged in a symmetric opposite position from the center of the arena and were then transported to their home cage. On day 3, the testing phase, mice were given 15 minutes to explore two objects: a familiar object and a novel one, in the same arena, keeping the same object localization.

The following behaviors were considered as exploration of the objects: sniffing, licking, or touching the object with the nose or with the front legs or directing the nose to the object at a distance ≤ 1 cm. Investigation was not scored if the mouse was on top of the object or completely immobile. The discrimination index was calculated as (time spent exploring the new object - time spent exploring the familiar object) / (total time spent exploring both objects). As a control, preference index for the (right/left) object location or for the object A versus B during the training phase of the novel object recognition (NOR) was measured in all groups of mice exposed to the test. We confirm here that no initial preference for any exposed object (A or B) or any orientation (right/left) was observed in any groups. The locomotion was assessed for each mouse. Behavior was scored on videos by two observers blind to treatment and the total exploration time of the objects was quantified in the testing phases.

### Novel Object Location (NOL)

For the novel object location task, all procedures were identical to the novel object recognition task except that during the testing phase, rather than presenting a novel object, mice encountered both familiar objects, with one object located in a different place in the arena. The time and frequency of exploration of the novel/relocated object is measured as an index of memory. Behavior was scored on videos by two observers blind to treatment and the total exploration time of the objects was quantified in the testing phases.

### Lisht-to-Dark Transition Test (D/LT)

This test is based on the innate aversion of rodents to brightly illuminated areas and on their spontaneous exploratory behavior in response to the stressor that light represents. The test apparatus consists of a dark, safe compartment and an illuminated, aversive one. The lit compartment was brightly illuminated with an 8 W fluorescent tube (1000 lx). Naive mice were placed individually in the testing chamber in the middle of the dark area facing away from the doorway to the light compartment. Mice were tested for 10 min, and two parameters were recorded: time spent in the lit compartment and the number of transitions between compartments, indices of anxiety-related behavior and exploratory activity. Behavior was scored using an infrared light beam activity monitor using actiMot2 Software (PhenoMaster Software, TSE).

### Open Field Test (OFT)

This test takes advantage of the aversion of rodents to brightly lit areas. Each mouse is placed in the center of the OFT chamber (43 × 43 cm chamber) and allowed to explore for 30 min. Mice were monitored throughout each test session by infrared light beam activity monitor using actiMot2 Software (PhenoMaster Software, TSE). The overall motor activity was quantified as the total distance travelled (ambulation). Anxiety was quantified by measuring the time and distance spent in the center versus periphery of the open-field chamber.

### Tail Suspension Test (TST)

This test is based on the observation that rodents, after initial escape-oriented movements, develop an immobile posture when placed in an inescapable stressful situation. Each mouse is hung in an uncontrollable fashion by their tail at above 25 cm from the floor. Mice were tested for 5 min, and the time spent immobile was quantified.

### Forced Swim Test (FST)

This test is based on a similar observation than the TST. Each mouse is placed in a cylinder (height: 25 cm and diameter: 10 cm) filled with water (23-25° C.). Mice were tested for 5 min, and the time spent immobile (behavioral despair) was quantified.

### EXAMPLE 1: CAN MODEL

### Clinical Features of Crouzon Syndrome With Acanthosis Nigricans

CAN syndrome, associated to a FGFR3 mutation, exhibits a skeletal phenotype similar to Crouzon syndrome [MIM 123500] due to FGFR2 mutations (Coll et al., 2018, 2016; Di Rocco et al., 2011): oculo-orbital disproportion, prognathism, midfacial hypoplasia (Data not shown), and brachycephaly, secondary to a premature fusion of the coronal and sagittal sutures (at various degrees) (Data not shown). Skull vault anomalies exert mechanical pressure on the brain and increase the risk for elevated intracranial pressure (Al-Namnam et al., 2019) (Data not shown). Additionally, brain MRI revealed mild temporal anomalies in three non-related CAN patients. Affected cases presented thickened parahippocampal groove, with modification of angle of the structure (Data not shown). One case presenting cloverleaf skull could not be interpreted.

Skull base anomalies in Crouzon syndrome patients, both FGFR2- and FGFR3-related, contribute anteriorly to midface hypoplasia and posteriorly to cranio-vertebral junction anomalies. Premature fusion of sphenooccipital synchondrosis is associated to shortened skull base, while premature fusion of intraoccipital synchondroses is associated with a narrowed foramen magnum in CAN patients (Data not shown).

### Skeletal Phenotype Is Mildly Affected in CAN Mouse Model Fgfr3A385E/+

To evaluate the effect of CAN mutation on the skeleton, we generated a mouse model expressing a ubiquitous p.Ala385Glu missense mutation corresponding to the p.Ala391Glu human mutation (Data not shown). The FGFR3 p.Ala385Glu transcripts were detected in fibroblasts and calvarial osteoblasts (Data not shown). Similarly to the human disease, no obvious limb shortening phenotype was observed in Fgfr3^{A385E/+} mice from antenatal and newborn stages (data not shown) to adult stage (Data not shown). Body weight, nasal-anal and femurs and tibias lengths were similar in Fgfr3^{A385E/+} and Fgfr3^{+/+} mice (Data not shown). Normal femur growth was confirmed by cartilage assessment in Fgfr3^{A385E/+} showing well organized growth plate without anomaly of the hypertrophic zone revealed with collagen type X staining (Data not shown). To confirm the absence of abnormal phenotype, bone structure parameters were assessed. Micro CT images of 3 months old femurs revealed a normal structure of the trabecular and cortical bone in Fgfr3A385E/+ mice (Data not shown).

The craniofacial phenotype was assessed using micro CT skull acquisition. The Fgfr3A385E/+ mice showed normal craniofacial features (Data not shown); the coronal sutures and skull base synchondroses were patent at post-natal day 21 similarly to control mice (Data not shown). Landmark-based geometric morphometrics (Heuzé et al., 2010) of Fgfr3^{A385E/+} mice and Fgfr3^{+/+} mice did not show any difference in skull shape (d = 0.0148; p = 0.0940). However, the shape of the mandible was significantly different between the two groups of mice (d=0.0159; P<0.01) (Data not shown). In order to confirm the absence of premature closure of the fronto-parietal suture, we assessed in vitro function of calvarial Fgfr3^{A385E/+} osteoblasts. No significant differences in mineralization capacity, proliferation and Mitogen-activated protein kinases (MAPK) activation were observed in the osteoblasts of Fgfr3^{A385E/+} mice compared to controls (Data not shown). All these data allowed us to conclude that p.Ala385Glu mutation expressed in osteoblasts was not active and consequently did not affect craniofacial development (Data not shown). These data explain the absence of craniosynostosis phenotype in Fgfr3^{A385E/+} mice. Regarding the skin, no signs of hyperkeratosis nor modification in thickness and pigmentation of the epidermis was detected in Fgfr3^{A385E/+} mice (Data not shown).

### Fgfr3A385E/+ Mouse Model Show Dentate Gyrus Decreased Neurogenesis

Structural brain anomalies have been described in CAN (Gürbüz et al., 2016) (Data not shown) and Muenke patients (Abdel-Salam et al., 2011; Grosso et al., 2003; Okubo et al., 2017) . These anomalies include abnormal morphology of hippocampus and temporal lobes. It is well known that the premature fusion of cranial sutures modifies the shape of the skull and impair the normal brain growth, causing functional issues such as increased intracranial pressure, visual impairment, deafness and cognitive impairment (Di Rocco et al., 2011). Previous studies showed that patients with MS presented deficits in adaptive and executive functions. This behavior phenotype included working memory deficits, attention-deficit hyperactivity disorder, emotional control and anxiety (Yarnell et al., 2015). These neurological impairments suggest an impact of FGFR3 in the brain for the control of cognitive functions. Hence, we performed magnetic resonance imaging (MRI) in Fgfr3^{A385E/+} mice at four months of age and measured the volumes of various brain regions.

No modifications of the volume and any compression were observed in the various brain regions of Fgfr3^{A385E/+} mice suggesting normal embryonic brain development (Data not shown). However, FGF and FGFR are known to be involved in proliferation and differentiation of neural stem cells and neural progenitors in the central nervous system (Huang et al., 2017; Kang and Hébert, 2015, Moldrich et al., 2011; Ohkubo et al., 2004; Stevens et al., 2012). Therefore, we hypothesized that the Fgfr3^{A385E} mutation expressed in the brain of Fgfr3^{A385E/+} mice could impact adult neurogenesis. The absence of craniofacial anomalies is a good opportunity to assess neurogenesis specifically during adult period. Exploring the role of Fgfr3 in adult neurogenesis, we observed a similar expression of FGFR3 in Fgfr3^{A385E/+} and Fgfr3^{+/+} mouse hippocampi by immunofluorescence (Data not shown) and western blotting (Data not shown). However, the canonical MAPK pathway activated by FGFR3 is found dysregulated in both Fgfr3 knock in mouse model (Komla-Ebri et al., 2016) and FGFR3 knock out mouse model (Zhou et al., 2015). Indeed, we observed a significant increased expression of phosphorylated Erk½ in adult hippocampi lysates (Data not shown) thus confirming that the Fgfr3^{A385E} mutation in the brain activated the MAPK pathway. FGFR3 play a key role in progenitor cell proliferation and neuronal differentiation in dentate gyrus (Inglis-Broadgate et al., 2005; Kang and Hébert, 2015; Moldrich et al., 2011; Thomson et al., 2009). In 4 months old Fgfr3^{A385E} hippocampi, using NeuN marker, we showed that mature neuron area of dentate gyrus granular layer is significantly decreased compared to control (Data not shown). We assessed whether this decreased neuronal population was caused by decreased progenitor proliferation. The positive cell number for the cell cycle KI67 marker was significantly reduced in the dentate gyrus subgranular zone in Fgfr3^{A385E/+} mice (Data not shown). In the granular zone, the neuronal differentiation rate revealed by doublecortin (DCX) immunolabelling is slightly decreased (Data not shown). Altogether these data strongly suggested that the Fgfr3 gain-of-function mutation affects predominantly proliferation thus impacting neuronal mature differentiation in dentate gyrus.

### Fgfr3A385E/+ Mouse Model Show Decreased Learning Capacity and Antidepressant Effect

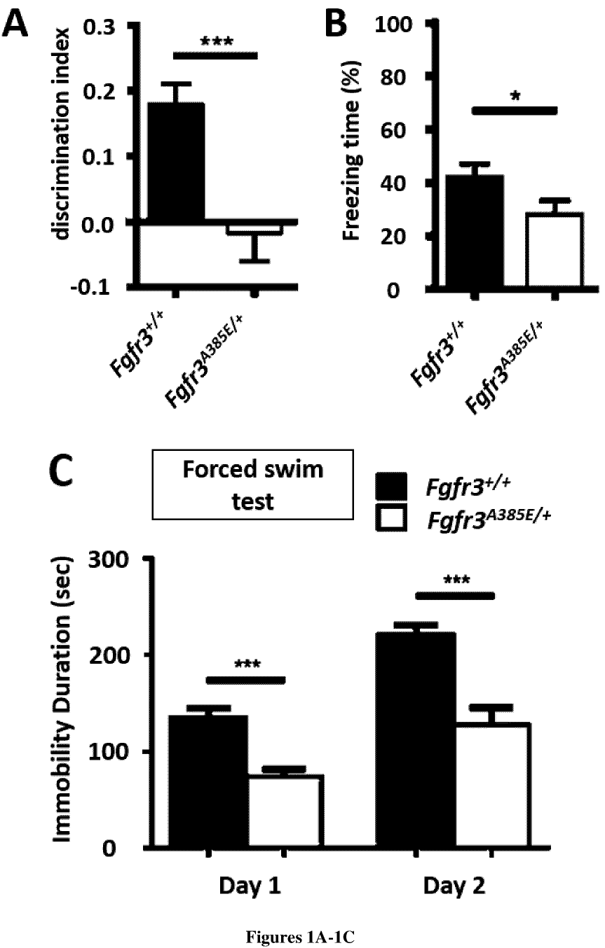

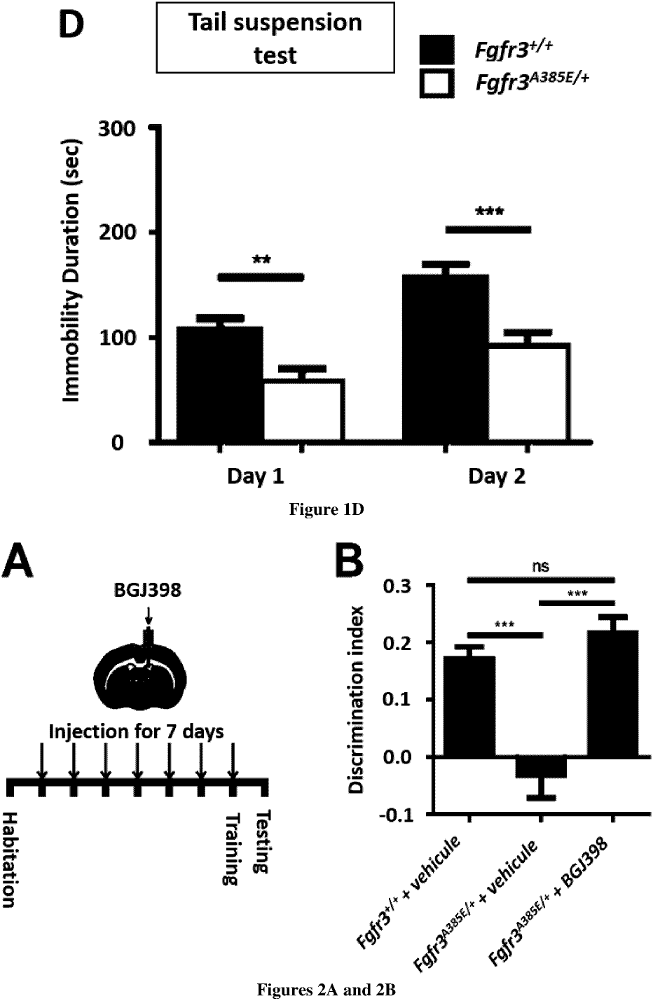

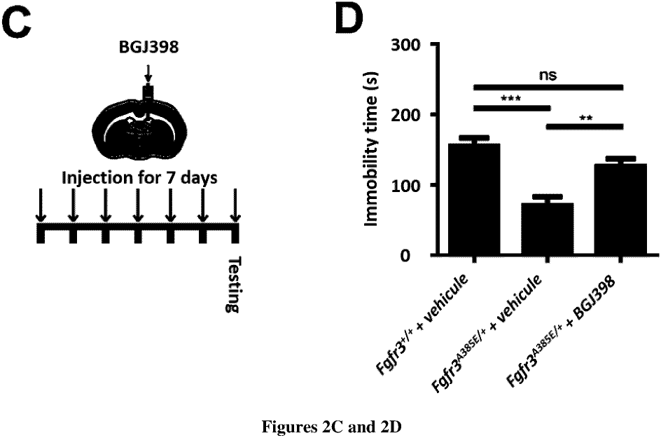

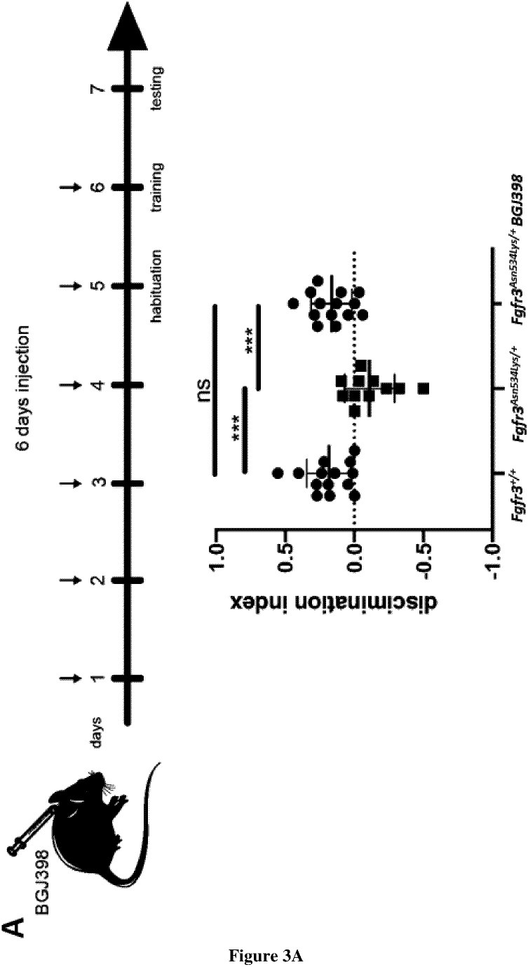

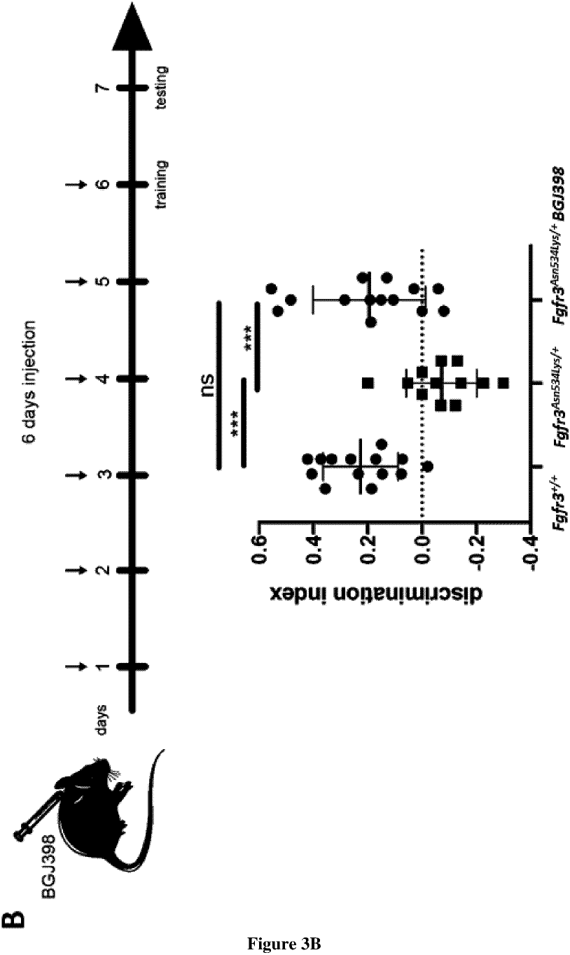

Reduced size of hippocampal structures and reduced proliferation were showed to be associated with memory and cognitive alterations in human and mouse (Kitamura and Inokuchi, 2014). Therefore, we subjected 4-month-old Fgfr3^{A385E/+} mice and their control littermates to a series of behavioral tests, that is thought to reflect behavioral functions related to hippocampus, measuring associative (one-trial contextual fear conditioning, CFC) and episodic (novel object recognition test, NOR), and spatial (Morris Water Maze, MWM) learning and memory (FIGS. 1A to 1D). In CFC, mutant mice exhibited no differences in baseline freezing time. However, Fgfr3 gain-of-function mutation resulted in decreased context-elicited freezing time during the testing phase compared to their control littermates, indicating that contextual fear memory is impaired in the mutant mice (FIG. 1B).

Next, we used a modified version of the NOR paradigm (Denny et al., 2012; Ennaceur and Delacour, 1988) that measures the rodent’s ability to recognize a novel object in the environment. Wild type mice are capable of differentiating novel objects from familiar ones and tend to explore novel ones for longer time. As shown in (FIG. 1A), 4-month-old Fgfr3^{A385E/+} mice explored significantly less the novel object than controls. However, no impairments were observed when memory was analyzed through the MWM task (assessing spatial learning and memory in rodents). Of note, Fgfr3^{A385E/+} mice and control had comparable performance in the open field test (OFT) and Light/Dark paradigm (L/DT) (Data not shown), indicating that their locomotion and anxious state were intact.

Next, we evaluated the coping strategy to an inescapable stress using the Forced Swim Test (FST) and the Tail suspension test (TST). As shown in (FIG. 1C), 4-month-old Fgfr3^{A385E/+} mice spend significantly less time immobile than control mice during the FST. The same impairments were observed during the TST. Indeed, mutant mice spend significantly less time immobile than control littermates (FIG. 1D).

Taken together, these data demonstrate that Fgfr3 gain-of-function mutation significantly influences hippocampal-dependent episodic memory and associative fear memory acquisition and affects the coping strategy to an inescapable stress.

Importantly, these data indicate that Fgfr3^{A385E} mutation in mice reproduced the behavioral deficits previously described in human patient, including working memory deficits, and emotional control and attention-deficit hyperactivity disorder (Yarnell et al., 2015).

### BGJ398 Intraventricular Injection Rescue the Cognitive Impairments of Fgfr3^{A385E/+} Mice

To confirm that cognitive impairments in Fgfr3^{A385E/+} mice were due to increased phosphorylation of the receptor, we decided to treat the mice with a specific tyrosine kinase inhibitor BGJ398 (Infigratinib) (Gudernova et al., 2015; Komla-Ebri et al., 2016). 4-month-old Fgfr3^{A385E/+} mice and their control littermates received intra-cerebro-ventricular injections for 7 days with BGJ398 or vehicle solution and were submitted to two behavioral tests (NOR and FST; FIGS. 2A to 2C). Injections of BGJ398 reversed the memory deficits observed in the NOR paradigm for the Fgfr3^{A385E/+} mice (FIG. 2B) and reestablished the coping strategy to an inescapable stress observed in the FST (FIG. 2D) compared to the control littermates. The absence of craniocerebral disproportion, and thus the lack of potentially increased intracranial hypertension in Fgfr3^{A385E/+} mice allow us to conclude that the reported behavioral anomalies observed were due to the direct impact of Fgfr3^{A385E} mutation on the brain. The rescue of these behavioral anomalies with the Fgfr3 inhibitor BGJ398 confirmed our hypotheses and supported the fact that FGFR3 over-activation was involved in the cognitive phenotype of patients with FGFR3-related craniosynostoses.

### EXAMPLE 2: HCH MODEL

### Fgfr3^{Asn534Lys/+} Mice Exhibit Brain Morphological Abnormalities

We previously described that Fgfr3^{Asn534Lys/+} mice show skulls anomalies with a large skull and premature fusion of skull base synchondrosis (Loisay et al manuscript in preparation). It was reported that brain anomalies and lobe dysgenesis are characteristics of Fgfr3 gain-of -function mutation mouse models. Therefore, we performed MRI and 3D reconstruction of Fgfr3^{Asn534Lys/+} mice and their control littermate brain. While we did not observed any significant abnormalities of hippocampal (p= 0.6905) and total brain volume (p= 0.3810) in Hch mice (Data not shown), MRI analyses showed modification of the brain shape when compared to controls (Data not shown).

### Learning and Memory Deficits in Fgfr3^{Asn534Lys/+} Mice

Adult neurogenesis is known to play an important role in the maintenance of hippocampal memory capacity. We subjected 4 months-old male Fgfr3^{Asn534Lys/+} mice and their control littermate to series of behavioral tests measuring spatial (novel object location, NOL) and episodic (novel object recognition, NOR) learning and memory. In NOR, we found that Hch mutant mice explored significantly less (p< <0.0001) the novel object than control littermates during the testing phase (Data not shown). The same impairments were observed when spatial memory was analyzed through the NOL test. Indeed, we found that Fgfr3^{Asn534Lys/+} mice explored significantly less the relocated object than control littermates during the testing phase (p<0.0001) (Data not shown).

### FGFR3 Tyrosine Kinase Inhibitor Treatment Is Sufficient to Reverse Cognitive Impairments Observed in Fgfr3^{Asn534Lys/+} Mice

Subcutaneous injections of BGJ398 (infigratinib), a tyrosine kinase inhibitor, is sufficient to abolished the FGFR3 over-activation and rescued the chondrodysplasia phenotype (Komla-Ebri et al. 2016). To confirm the impact of FGFR3 gain-of-function in learning impairment, we treated Fgfr3^{Asn534Lys/+} mice during six days with BGJ398. As a result, we found that injections of BGJ398 are sufficient to restore the spatial (NOL) (FIG. 3A) and episodic memory deficits (NOR) (FIG. 3B) observed in Fgfr3^{Asn534Lys/+} mice. Indeed, injected mutant mice were able to explore the relocated object (NOL) (p= 0.9538) and the novel object (NOR) (p= 0.8697) to the same levels than control littermates (p= 0.9538) (FIGS. 3A and 3B). Taken together these results confirm the importance of FGFR3 in the regulation of learning and memory.

### Stress and Memory Behavior Test

We next perform one-trial contextual fear conditioning (CFC) on 4-month-old male mice to assess associative memory. While, Fgfr3^{Asn534Lys/+} mice did not show any impairment in baseline freezing time (p= 0.2303), surprisingly mutant mice show significant increased in context elicited freezing time compared to the control mice during the testing phase (p= 0.0466). This result strongly suggests that contextual fear memory is exacerbated in the mutant mice.

### Anti-Depressant Effect of FGFR3

FGF pathway might be involved in depression disorders. Therefore, we performed Tail suspension test (TST) and force swim test (FST) in Fgfr3^{Asn534Lys/+} mice (Data not shown). Animals subjected to the short-term and inescapable stress of being suspended by their tails or forced to swim will develop an immobile posture characteristic of depression-related behavior that will be scored. Performing these tests, we found that, either in TST or FST, Fgfr3^{Asn534Lys/+} mice present a reduction in immobile posture, suggesting that gain-of-function mutation in FGFR3 may have anti-depressant effects.

### No Effect in Anxiety Behavior in Fgfr3^{Asn534Lys/+} Mice

Mutant and WT mice had comparable performance in the open field test (OFT) (Data not shown) and Light/Dark paradigm (L/DT) (Data not shown), indicating that their locomotion and anxious state were unaffected. Of a note, due to the dwarf phenotype of Fgfr3^{Asn534Lys/+} mice, the mutant mice were slower than control littermates (p=0.0003) and the total distance traveled during OF is reduced (p=0.0003). Consequently, OFT data analysis was performed considering the % of distance (distance in center/distance total*100). Taken together these results indicate that FGFR3 do not play a key role in anxiety and confirm that the cognitive defects observed for learning and memory in our mutant mice are independent to any anxiety- or exploratory-related behavioural defects..

### Discussion

CAN syndrome is a very rare syndromic craniosynostosis associated to the specific p.Ala391Glu gain-of-function mutation in FGFR3 (Meyers et al., 1995). The effect of the p.Ala391Glu mutation was previously described as causing overactivation of FGFR3 (Chen et al., 2013, 2011; Li et al., 2006).

Mouse Fgfr3^{A385E/+} CAN model showed an absence of major craniofacial skeletal phenotype. Interestingly, the phenotype of Fgfr3^{A385E/+} was comparable to the one observed in Fgfr3^{P244R/P244R} mice, a mouse model for Muenke syndrome. Suture and synchondroses were found to be mildly affected in Muenke Fgfr3^{P244R/P244R} mutants, with coronal suture fused in a minority of individuals (Laurita et al., 2011; Twigg et al., 2008).

In CAN and Muenke, premature fusion of skull vault sutures combined with premature fusion of skull base synchondroses associated to narrowing of foramen magnum lead to increased intracranial pressure in patients (Di Rocco et al., 2011). Premature fusion of the skull vault is also associated with brain structures anomalies, including abnormal hippocampus development (Grosso et al., 2003; Gürbüz et al., 2016; Okubo et al., 2017).

HCH patients (14600 MIM) are characterized by rhizomelic dwarfism, mild macrocephaly, hypoplasia of the midface, short square ilia and in some case acanthosis nigricans (Blomberg et al. 2010). The most common HCH mutation (p.Asn540Lys) is localized in tyrosine kinase 1 domain of FGFR3 (Bonaventure et al. 1996; Rousseau et al. 1994).

HCH patients present lobe dysgenesis (Kannu et al. 2005) and abnormal hippocampus configuration (Linnankivi et al. 2012). Moreover, HCH patients present learning impairment, mild intellectual disability, global development delay and occasionally seizure and epilepsy (Linnankivi et al. 2012)

In Fgfr3 mouse models, previous studies on mouse model Fgfr3^{+/K644E} mutation associated to thanatophoric dysplasia, both ubiquitously and under Nestin promoter, presented severe overgrowth of cerebrum and cortex, while Fgfr3^{-/-} mouse presented an underdeveloped neocortex (Inglis-Broadgate et al., 2005; Moldrich et al., 2011; Thomson et al., 2009, 2007). It is commonly accepted that the premature fusion of cranial sutures observed in craniosynostoses modifies brain morphology and is associated with cognitive impairment via a chronic increase in intracranial pressure (Aldridge et al., 2010; Arnaud-López et al., 2007; Gürbüz et al., 2016; Martínez-Abadías et al., 2011). Here, taking advantage of the absence of abnormal skull phenotype in the Fgfr3^{A385E/+} mice, we analyzed the role of activating Fgfr3 gain-of-function mutation in the brain. The brain of Fgfr3^{A385E/+} mice did not present severe morphological modifications, thus indicating that Fgfr3^{A385E} mutation had a moderate impact on brain embryonic neurogenesis. By contrast, the analysis of adult hippocampal neurogenesis showed decreased progenitor proliferation in the dentate gyrus, supported by decreased granular zone of the dentate gyrus.

Interestingly, previous studies reported decreased progenitor proliferation in FGFR1,2,3 loss-of-function mutations, while over-activation of FGFR3 (Fgfr3^{TDIIK650E}) promote increased progenitor differentiation in the dentate gyrus (Kang and Hébert, 2015). Our results contrast with these observations. We observed that an over-activation of FGFR3 led to decreased cell proliferation in Fgfr3^{A385E/+} mice. It seemed that the level of phosphorylation of the receptor interfered with neurogenesis: Fgfr3^{TDIIK650E} mutation led to the excessive receptor activation levels whereas Fgfr3^{A385E} mutation led to a more moderate over-activation. These data suggested that FGFR regulation of hippocampal neurogenesis is linked to the level of FGFR3 activation.

We next analyzed the impact of Fgfr3^{A385E} and of Fgfr3^{N534K} mutation on brain cognitive functions. We observed that Fgfr3^{A385E/+} mice Fgfr3N^{534K/+} mice exhibited severe impairments working and episodic memory functions without any locomotion, anxiety-related behavioral or spatial memory phenotype. While, the effect of FGFR signals on memory is unclear, to date, our study is the first to associate Fgfr3 mutation to cognitive abnormalities in mice. Mechanistically, the deficit in learning and memory performances could be linked, at least in part, to the decreased hippocampal neurogenesis observed in our mutant mice. Interestingly, the deletion of Fgfr2 in embryo and adult mice showed decreased progenitor proliferation and differentiation in the dentate gyrus with specific negative effect on associative and spatial memory capacity (Stevens et al., 2012). Collectively, our data demonstrate that Fgfr3 gain-of-function mutation lead to severe learning and memory deficits and lower adult neurogenesis in the hippocampus.

In addition, a decreased coping strategy to an inescapable stress (previously name “depression like behavior”) was observed in Fgfr3^{A385E/+} mice and Fgfr3^{N534K/+} mice. This role of FGFR3 was previously reported in major depressive disorders in human associated with downregulation of the receptor (Evans et al., 2004). Furthermore, patients with FGFR3 related craniosynostosis syndromes, showed an impairment of emotional control and anxiety behavior (de Jong et al., 2010; Maliepaard et al., 2014; Yarnell et al., 2015). Today, no study reported depressive or mood disorders in craniosynostoses cases. Animal models studies also demonstrated the antidepressant and anxiolytic effect of FGF2 in FGF2 knockout or exogenous FGF2 injections (Elsayed et al., 2012; Salmaso et al., 2016). In contrast to FGF2, FGF9 plays a depressive effect in mouse (Aurbach et al., 2015). Both FGF2 and FGF9 are among the major FGFR3 ligands and can also bind the other FGFRs. These observations are in agreement with our data and could involve a complex combination of different FGFs and FGFR1, 2, 3 bindings. However, it is unclear how the antidepressant phenotype observed in Fgfr3^{A385E/+} mice and Fgfr3^{N534K/+} mice could be translated in human condition.

To confirm the direct implication of brain FGFR3 in cognitive disorders observed in Fgfr3^{A385E/+} mice and Fgfr3^{N534K/+} mice, we decided to treat mice with BGJ398, a tyrosine kinase inhibitor, following selective brain injections. BGJ398 was selected for its highest binding specificity for FGFR3 and a previous work showed the efficacy of BGJ398 in skeletal anomalies in an FGFR3 related Achondroplasia mouse model (Komla-Ebri et al., 2016). Here, intra-cerebro-ventricular injection of BGJ398 in adult Fgfr3^{A385E/+} mice and Fgfr3N^{534K/+} mice showed rescue of the working and episodic memory deficits and antidepressant effect. These data demonstrated the direct impact of the Fgfr3 gain-of-function mutation in the brain on cognitive performances. We also demonstrated that FGFR3 played a major role in hippocampal adult neurogenesis and we established a direct link between hippocampal anomalies and learning and stress response.

These results highlighted the presence of cognitive impairments without a phenotype of craniosynostosis in a mouse model expressing Fgfr3 gain-of -function mutation. Our data suggest that the brain of patients with FGFR-related craniosynostosis can be affected by the mutation independently of skull anomalies.

### REFERENCES

Throughout this application, various references describe the state of the art to which this invention pertains. The disclosures of these references are hereby incorporated by reference into the present disclosure.
Abdel-Salam, G.M.H., Flores-Sarnat, L., El-Ruby, M.O., Parboosingh, J., Bridge, P., Eid, M.M., El-Badry, T.H., Effat, L., Curatolo, P., Temtamy, S.A., 2011. Muenke syndrome with pigmentary disorder and probable hemimegalencephaly: An expansion of the phenotype. Am. J. Med. Genet. 155, 207-214. https://doi.org/10.1002/ajmg.a.33777
Aldridge, K., Hill, C.A., Austin, J.R., Percival, C., Martinez-Abadias, N., Neuberger, T., Wang, Y., Jabs, E.W., Richtsmeier, J.T., 2010. Brain phenotypes in two FGFR2 mouse models for Apert syndrome. Dev. Dyn. 239, 987-997. https://doi.org/10.1002/dvdy.22218
Al-Namnam, N.M., Hariri, F., Thong, M.K., Rahman, Z.A., 2019. Crouzon syndrome: Genetic and intervention review. J Oral Biol Craniofac Res 9, 37-39. https://doi.org/10.1016/j.jobcr.2018.08.007
Arnaud-López, L., Fragoso, R., Mantilla-Capacho, J., Barros-Núñez, P., 2007. Crouzon with acanthosis nigricans. Further delineation of the syndrome. Clin. Genet. 72, 405-410. https://doi.org/10.1111/j.1399-0004.2007.00884.x
Aurbach, E.L., Inui, E.G., Turner, C.A., Hagenauer, M.H., Prater, K.E., Li, J.Z., Absher, D., Shah, N., Blandino, P., Bunney, W.E., Myers, R.M., Barchas, J.D., Schatzberg, A.F., Watson, S.J., Akil, H., 2015. Fibroblast growth factor 9 is a novel modulator of negative affect. Proc Natl Acad Sci U S A 112, 11953-11958. https://doi.org/10.1073/pnas.1510456112
Berton, Olivier, Colleen A. McClung, Ralph J. DiLeone, Vaishnav Krishnan, William Renthal, Scott J. Russo, Danielle Graham, et al. 2006. « Essential Role of BDNF in the Mesolimbic Dopamine Pathway in Social Defeat Stress ». Science 311 (5762): 864-68. https://doi.org/10.1126/science.1120972.
Blomberg, M., E. M. Jeppesen, F. Skovby, et E. Benfeldt. 2010. « FGFR3 Mutations and the Skin: Report of a Patient with a FGFR3 Gene Mutation, Acanthosis Nigricans, Hypochondroplasia and Hyperinsulinemia and Review of the Literature ». Dermatology 220 (4): 297-305. https://doi.org/10.1159/000297575.
Bonaventure, J., F. Rousseau, L. Legeai-Mallet, M. Le Merrer, A. Munnich, et P. Maroteaux. 1996. « Common Mutations in the Fibroblast Growth Factor Receptor 3 (FGFR3) Gene Account for Achondroplasia, Hypochondroplasia, and Thanatophoric Dwarfism ». American Journal of Medical Genetics 63 (1): 148-54. https://doi.org/10.1002/(SICI)1096-8628(19960503)63:1<148::AID-AJMG26>3.0.CO;2-N.
Chen, F., Degnin, C., Laederich, M., Horton, W., Hristova, K., 2011. The A391E mutation enhances FGFR3 activation in the absence of ligand. Biochim Biophys Acta 1808, 2045-2050. https://doi.org/10.1016/j.bbamem.2011.04.007
Chen, F., Sarabipour, S., Hristova, K., 2013. Multiple Consequences of a Single Amino Acid Pathogenic RTK Mutation: The A391E Mutation in FGFR3. PLoS One 8. https://doi.org/10.1371/journal.pone.0056521
Coll, G., Lemaire, J.-J., Di Rocco, F., Barthélémy, I., Garcier, J.-M., De Schlichting, E., Sakka, L., 2016. Human Foramen Magnum Area and Posterior Cranial Fossa Volume Growth in Relation to Cranial Base Synchondrosis Closure in the Course of Child Development: Neurosurgery 79, 722-735. https://doi.org/10.1227/NEU.0000000000001309
Coll, G., Sakka, L., Botella, C., Pham-Dang, N., Collet, C., Zerah, M., Arnaud, E., Di Rocco, F., 2018. Pattern of Closure of Skull Base Synchondroses in Crouzon Syndrome. World Neurosurgery 109, e460-e467. https://doi.org/10.1016/j.wneu.2017.09.208
Colvin, J.S., Bohne, B.A., Harding, G.W., McEwen, D.G., Omitz, D.M., 1996. Skeletal overgrowth and deafness in mice lacking fibroblast growth factor receptor 3. Nat. Genet. 12, 390-397. https://doi.org/10.1038/ng0496-390
Commons, K.G., Cholanians, A.B., Babb, J.A., Ehlinger, D.G., 2017. The Rodent Forced Swim Test Measures Stress-Coping Strategy, Not Depression-like Behavior. ACS Chem Neurosci 8, 955-960. https://doi.org/10.1021/acschemneuro.7b00042
Cornille, Maxence, Stéphanie Moriceau, Roman Hossein Khonsari, Yann Heuzé, Anne Morice, Eric Arnaud, Corinne Collet, et al. 2020. « FGFR3 gain-of-function is associated to memory and learning deficit in Crouzon syndrome mouse model », under submission 2020.
de Jong, T., Bannink, N., Bredero-Boelhouwer, H.H., van Veelen, M.L.C., Bartels, M.C., Hoeve, L.J., Hoogeboom, A.J.M., Wolvius, E.B., Lequin, M.H., van der Meulen, J.J.N.M., van Adrichem, L.N.A., Vaandrager, J.M., Ongkosuwito, E.M., Joosten, K.F.M., Mathijssen, I.M.J., 2010. Long-term functional outcome in 167 patients with syndromic craniosynostosis; defining a syndrome-specific risk profile. J Plast Reconstr Aesthet Surg 63, 1635-1641. https://doi.org/10.1016/j.bjps.2009.10.029
Deng, C., Wynshaw-Boris, A., Zhou, F., Kuo, A., Leder, P., 1996. Fibroblast growth factor receptor 3 is a negative regulator of bone growth. Cell 84, 911-921.
Denny, C.A., Burghardt, N.S., Schachter, D.M., Hen, R., Drew, M.R., 2012. 4- to 6-week-old adult-born hippocampal neurons influence novelty-evoked exploration and contextual fear conditioning. Hippocampus 22, 1188-1201. https://doi.org/10.1002/hipo.20964
Di Rocco, F., Biosse Duplan, M., Heuzé, Y., Kaci, N., Komla-Ebri, D., Munnich, A., Mugniery, E., Benoist-Lasselin, C., Legeai-Mallet, L., 2014. FGFR3 mutation causes abnormal membranous ossification in achondroplasia. Hum. Mol. Genet. 23, 2914-2925. https://doi.org/10.1093/hmg/ddu004
Di Rocco, F., Collet, C., Legeai-Mallet, L., Arnaud, E., Le Merrer, M., Hadj-Rabia, S., Renier, D., 2011. Crouzon syndrome with acanthosis nigricans: a case-based update. Childs Nerv Syst 27, 349-354. https://doi.org/10.1007/s00381-010-1347-z
Elsayed, M., Banasr, M., Duric, V., Fournier, N.M., Licznerski, P., Duman, R.S., 2012. Antidepressant effects of Fibroblast Growth Factor-2 in behavioral and cellular models of depression. Biol Psychiatry 72, 258-265. https://doi.org/10.1016/j.biopsych.2012.03.003
Ennaceur, A., Delacour, J., 1988. A new one-trial test for neurobiological studies of memory in rats. 1: Behavioral data. Behav. Brain Res. 31, 47-59.
Evans, S.J., Choudary, P.V., Neal, C.R., Li, J.Z., Vawter, M.P., Tomita, H., Lopez, J.F., Thompson, R.C., Meng, F., Stead, J.D., Walsh, D.M., Myers, R.M., Bunney, W.E., Watson, S.J., Jones, E.G., Akil, H., 2004. Dysregulation of the fibroblast growth factor system in major depression. Proc Natl Acad Sci U S A 101, 15506-15511. https://doi.org/10.1073/pnas.0406788101
Gibbs, L., Legeai-Mallet, L., 2007. FGFR3 intracellular mutations induce tyrosine phosphorylation in the Golgi and defective glycosylation. Biochimica et Biophysica Acta (BBA) - Molecular Cell Research 1773, 502-512. https://doi.org/10.1016/j.bbamcr.2006.12.010
Glatigny, M., Moriceau, S., Rivagorda, M., Ramos-Brossier, M., Nascimbeni, A.C., Lante, F., Shanley, M.R., Boudarene, N., Rousseaud, A., Friedman, A.K., Settembre, C., Kuperwasser, N., Friedlander, G., Buisson, A., Morel, E., Codogno, P., Oury, F., 2019. Autophagy Is Required for Memory Formation and Reverses Age-Related Memory Decline. Curr. Biol. https://doi.org/10.1016/j.cub.2018.12.021
Grosso, S., Farnetani, M.A., Berardi, R., Bartalini, G., Carpentieri, M., Galluzzi, P., Mostardini, R., Morgese, G., Balestri, P., 2003. Medial temporal lobe dysgenesis in Muenke syndrome and hypochondroplasia. American Journal of Medical Genetics Part A 120A, 88-91. https://doi.org/10.1002/ajmg.a.10171
Gudernova, I., Vesela, I., Balek, L., Buchtova, M., Dosedelova, H., Kunova, M., Pivnicka, J., Jelinkova, I., Roubalova, L., Kozubik, A., Krejci, P., 2015. Multikinase activity of fibroblast growth factor receptor (FGFR) inhibitors SU5402, PD173074, AZD1480, AZD4547 and BGJ398 compromises the use of small chemicals targeting FGFR catalytic activity for therapy of short stature syndromes. Hum. Mol. Genet. https://doi.org/10.1093/hmg/ddv441
Gürbüz, F., Ceylaner, S., Topaloǧlu, A.K., Yüksel, B., 2016. Crouzonodermoskeletal Syndrome with Hypoplasia of Corpus Callosum and Inferior Vermis. J Clin Res Pediatr Endocrinol 8, 373-374. https://doi.org/10.4274/jcrpe.3343
Heuzé, Y., Boyadjiev, S.A., Marsh, J.L., Kane, A.A., Cherkez, E., Boggan, J.E., Richtsmeier, J.T., 2010. New insights into the relationship between suture closure and craniofacial dysmorphology in sagittal nonsyndromic craniosynostosis. J Anat 217, 85-96. https://doi.org/10.1111/j.1469-7580.2010.01258.x
Huang, J.-Y., Lynn Miskus, M., Lu, H.-C., 2017. FGF-FGFR Mediates the Activity-Dependent Dendritogenesis of Layer IV Neurons during Barrel Formation. J. Neurosci. 37, 12094-12105. https://doi.org/10.1523/JNEUROSCI.1174-17.2017
Imai, Yoshinori, et Shinichi Kohsaka. 2002. « Intracellular Signaling in M-CSF-Induced Microglia Activation: Role of Iba1 ». Glia 40 (2): 164-74. https://doi.org/10.1002/glia.10149.
Inglis-Broadgate, S.L., Thomson, R.E., Pellicano, F., Tartaglia, M.A., Pontikis, C.C., Cooper, J.D., Iwata, T., 2005. FGFR3 regulates brain size by controlling progenitor cell proliferation and apoptosis during embryonic development. Developmental Biology 279, 73-85. https://doi.org/10.1016/j.ydbio.2004.11.035
Itoh, Kyoko, Ritsuko Pooh, Yonehiro Kanemura, Mami Yamasaki, et Shinji Fushiki. 2013. « Brain Malformation with Loss of Normal FGFR3 Expression in Thanatophoric Dysplasia Type I ». Neuropathology 33 (6): 663-66. https://doi.org/10.1111/neup.12036.
Kang, W., Hébert, J.M., 2015. FGF Signaling Is Necessary for Neurogenesis in Young Mice and Sufficient to Reverse Its Decline in Old Mice. J Neurosci 35, 10217-10223. https://doi.org/10.1523/JNEUROSCI.1469-15.2015
Kannu, Peter, Ian M. Hayes, Simone Mandelstam, Leo Donnan, et Ravi Savarirayan. 2005. « Medial Temporal Lobe Dysgenesis in Hypochondroplasia ». American Journal of Medical Genetics. Part A 138 (4): 389-91. https://doi.org/10.1002/ajmg.a.30974.
Kitamura, T., Inokuchi, K., 2014. Role of adult neurogenesis in hippocampal-cortical memory consolidation. Mol Brain 7, 13. https://doi.org/10.1186/1756-6606-7-13
Klingenberg, C.P., 2011. MorphoJ: an integrated software package for geometric morphometrics. Mol Ecol Resour 11, 353-357. https://doi.org/10.1111/j.1755-0998.2010.02924.x
Komla-Ebri, D., Dambroise, E., Kramer, I., Benoist-Lasselin, C., Kaci, N., Le Gall, C., Martin, L., Busca, P., Barbault, F., Graus-Porta, D., Munnich, A., Kneissel, M., Di Rocco, F., Biosse-Duplan, M., Legeai-Mallet, L., 2016. Tyrosine kinase inhibitor NVP-BGJ398 functionally improves FGFR3-related dwarfism in mouse model. J. Clin. Invest. 126, 1871-1884. https://doi.org/10.1172/JCI83926
Kruszka, P., Addissie, Y.A., Agochukwu, N.B., Doherty, E.S., Muenke, M., 1993. Muenke Syndrome, in: Adam, M.P., Ardinger, H.H., Pagon, R.A., Wallace, S.E., Bean, L.J., Stephens, K., Amemiya, A. (Eds.), GeneReviews®. University of Washington, Seattle, Seattle (WA).
Laurita, J., Koyama, E., Chin, B., Taylor, J.A., Lakin, G.E., Hankenson, K.D., Bartlett, S.P., Nah, H.-D., 2011. The Muenke syndrome mutation (FgfR3P244R) causes cranial base shortening associated with growth plate dysfunction and premature perichondrial ossification in murine basicranial synchondroses. Dev. Dyn. 240, 2584-2596. https://doi.org/10.1002/dvdy.22752
Li, E., You, M., Hristova, K., 2006. FGFR3 dimer stabilization due to a single amino acid pathogenic mutation. J. Mol. Biol. 356, 600-612. https://doi.org/10.1016/j.jmb.2005.11.077
Linnankivi, Tarja, Outi Mäkitie, Leena Valanne, et Sanna Toiviainen-Salo. 2012. « Neuroimaging and Neurological Findings in Patients with Hypochondroplasia and FGFR3 N540K Mutation ». American Journal of Medical Genetics. Part A 158A (12): 3119-25. https://doi.org/10.1002/ajmg.a.35642.
Manikkam, S. A., K. Chetcuti, K. B. Howell, R. Savarirayan, A. M. Fink, et S. A. Mandelstam. 2018. « Temporal Lobe Malformations in Achondroplasia: Expanding the Brain Imaging Phenotype Associated with FGFR3-Related Skeletal Dysplasias ». American Journal of Neuroradiology 39 (2): 380-84. https://doi.org/10.3174/ajnr.A5468.
Maliepaard, M., Mathijssen, I.M.J., Oosterlaan, J., Okkerse, J.M.E., 2014. Intellectual, Behavioral, and Emotional Functioning in Children With Syndromic Craniosynostosis. Pediatrics 133, e1608-e1615. https://doi.org/10.1542/peds.2013-3077
Marfínez-Abadías, N., Heuzé, Y., Wang, Y., Jabs, E.W., Aldridge, K., Richtsmeier, J.T., 2011. FGF/FGFR Signaling Coordinates Skull Development by Modulating Magnitude of Morphological Integration: Evidence from Apert Syndrome Mouse Models. PLoS One 6. https://doi.org/10.1371/journal.pone.0026425
Merrill, A.E., Sarukhanov, A., Krejci, P., Idoni, B., Camacho, N., Estrada, K.D., Lyons, K.M., Deixler, H., Robinson, H., Chitayat, D., Curry, C.J., Lachman, R.S., Wilcox, W.R., Krakow, D., 2012. Bent bone dysplasia-FGFR2 type, a distinct skeletal disorder, has deficient canonical FGF signaling. Am. J. Hum. Genet. 90, 550-557. https://doi.org/10.1016/j.ajhg.2012.02.005
Meyers, G.A., Orlow, S.J., Munro, I.R., Przylepa, K.A., Jabs, E.W., 1995. Fibroblast growth factor receptor 3 (FGFR3) transmembrane mutation in Crouzon syndrome with acanthosis nigricans. Nat. Genet. 11, 462-464. https://doi.org/10.1038/ng1295-462
Mir A, Wu T, Orlow SJ, 2013. CUtaneous features of crouzon syndrome with acanthosis nigricans. JAMA Dermatol 149, 737-741. https://doi.org/10.1001/jamadermatol.2013.3019
Moldrich, R.X., Mezzera, C., Holmes, W.M., Goda, S., Brookfield, S.J., Rankin, A.J., Barr, E., Kurniawan, N., Dewar, D., Richards, L.J., López-Bendito, G., Iwata, T., 2011. Fgfr3 regulates development of the caudal telencephalon. Developmental Dynamics 240, 1586-1599. https://doi.org/10.1002/dvdy.22636
Muenke, M., Gripp, K.W., McDonald-McGinn, D.M., Gaudenz, K., Whitaker, L.A., Bartlett, S.P., Markowitz, R.I., Robin, N.H., Nwokoro, N., Mulvihill, J.J., Losken, H.W., Mulliken, J.B., Guttmacher, A.E., Wilroy, R.S., Clarke, L.A., Hollway, G., Adès, L.C., Haan, E.A., Mulley, J.C., Cohen, M.M., Bellus, G.A., Francomano, C.A., Moloney, D.M., Wall, S.A., Wilkie, A.O.M., Zackai, E.H., 1997. A unique point mutation in the fibroblast growth factor receptor 3 gene (FGFR3) defines a new craniosynostosis syndrome. Am J Hum Genet 60, 555-564.
Neben, C.L., Idoni, B., Salva, J.E., Tuzon, C.T., Rice, J.C., Krakow, D., Merrill, A.E., 2014. Bent bone dysplasia syndrome reveals nucleolar activity for FGFR2 in ribosomal DNA transcription. Hum. Mol. Genet. 23, 5659-5671. https://doi.org/10.1093/hmg/ddu282
Neben, C.L., Tuzon, C.T., Mao, X., Lay, F.D., Merrill, A.E., 2017. FGFR2 mutations in bent bone dysplasia syndrome activate nucleolar stress and perturb cell fate determination. Hum. Mol. Genet. 26, 3253-3270. https://doi.org/10.1093/hmg/ddx209
Numakawa, Tadahiro, Haruki Odaka, Naoki Adachi, Shuichi Chiba, Yoshiko Ooshima, Hitomi Matsuno, Shingo Nakajima, et al. 2018. « Basic Fibroblast Growth Factor Increased Glucocorticoid Receptors in Cortical Neurons through MAP Kinase Pathway ».
Okubo, Y., Kitamura, T., Anzai, M., Endo, W., Inui, T., Takezawa, Y., Suzuki-Muromoto, S., Miyabayashi, T., Togashi, N., Oba, H., Saitsu, H., Matsumoto, N., Haginoya, K., 2017. A patient with Muenke syndrome manifesting migrating neonatal seizures. Brain Dev. https://doi.org/10.1016/j.braindev.2017.05.007
Richtsmeier, J.T., Flaherty, K., 2013. Hand in glove: brain and skull in development and dysmorphogenesis. Acta Neuropathol. 125, 469-489. https://doi.org/10.1007/s00401-013-1104-y
Robin, N.H., Falk, M.J., Haldeman-Englert, C.R., 1993. FGFR-Related Craniosynostosis Syndromes, in: Adam, M.P., Ardinger, H.H., Pagon, R.A., Wallace, S.E., Bean, L.J., Stephens, K., Amemiya, A. (Eds.), GeneReviews®. University of Washington, Seattle, Seattle (WA).
Rousseau, F., Bonaventure, J., Legeai-Mallet, L., Pelet, A., Rozet, J.M., Maroteaux, P., Le Merrer, M., Munnich, A., 1994. Mutations in the gene encoding fibroblast growth factor receptor-3 in achondroplasia. Nature 371, 252-254. https://doi.org/10.1038/371252a0
Salmaso, N., Stevens, H.E., McNeill, J., ElSayed, M., Ren, Q., Maragnoli, M.E., Schwartz, M.L., Tomasi, S., Sapolsky, R.M., Duman, R., Vaccarino, F.M., 2016. Fibroblast Growth Factor 2 Modulates Hypothalamic Pituitary Axis Activity and Anxiety Behavior Through Glucocorticoid Receptors. Biological Psychiatry, New Insight Into Depression Therapeutics 80, 479-489. https://doi.org/10.1016/j.biopsych.2016.02.026
Stevens, H.E., Jiang, G.Y., Schwartz, M.L., Vaccarino, F.M., 2012. Learning and memory depend on fibroblast growth factor receptor 2 functioning in hippocampus. Biol Psychiatry 71, 1090-1098. https://doi.org/10.1016/j.biopsych.2012.03.013
Su, N., Xu, X., Li, Cuiling, He, Q., Zhao, L., Li, Can, Chen, S., Luo, F., Yi, L., Du, X., Huang, H., Deng, C., Chen, L., 2010. Generation of Fgfr3 conditional knockout mice. Int. J. Biol. Sci. 6, 327-332.
Tang, Ming-ming, Wen-juan Lin, Yu-qin Pan, et Ying-cong Li. 2018. « Fibroblast Growth Factor 2 Modulates Hippocampal Microglia Activation in a Neuroinflammation Induced Model of Depression ». Frontiers in Cellular Neuroscience 12 (août). https://doi.org/10.3389/fncel.2018.00255.
Thomson, R.E., Kind, P.C., Graham, N.A., Etherson, M.L., Kennedy, J., Fernandes, A.C., Marques, C.S., Hevner, R.F., Iwata, T., 2009. Fgf receptor 3 activation promotes selective growth and expansion of occipitotemporal cortex. Neural Dev 4, 4. https://doi.org/10.1186/1749-8104-4-4
Thomson, R.E., Pellicano, F., Iwata, T., 2007. Fibroblast growth factor receptor 3 kinase domain mutation increases cortical progenitor proliferation via mitogen-activated protein kinase activation. Journal of Neurochemistry 100, 1565-1578. https://doi.org/10.1111/j.1471-4159.2006.04285.x
Twigg et al., 2008. Skeletal Analysis of the Fgfr3P244R Mouse, a Genetic Model for the Muenke Craniosynostosis Syndrome.
Twigg, S.R.F., Wilkie, A.O.M., 2015. A Genetic-Pathophysiological Framework for Craniosynostosis. American Journal of Human Genetics 97, 359. https://doi.org/10.1016/j.ajhg.2015.07.006
Wang, Lin, Xi-Xi Li, Xi Chen, Xiao-Yan Qin, Elissavet Kardami, et Yong Cheng. 2018. « Antidepressant-Like Effects of Low- and High-Molecular Weight FGF-2 on Chronic Unpredictable Mild Stress Mice ». Frontiers in Molecular Neuroscience 11 (octobre). https://doi.org/10.3389/fnmol.2018.00377.
Wilkie, A.O.M., Byren, J.C., Hurst, J.A., Jayamohan, J., Johnson, D., Knight, S.J.L., Lester, T., Richards, P.G., Twigg, S.R.F., Wall, S.A., 2010. Prevalence and complications of single gene and chromosomal disorders in craniosynostosis. Pediatrics 126, e391-e400. https://doi.org/10.1542/peds.2009-3491
Yarnell, C.M.P., Addissie, Y.A., Hadley, D.W., Guillen Sacoto, M.J., Agochukwu, N.B., Hart, R.A., Wiggs, E.A., Platte, P., Paelecke, Y., Collmann, H., Schweitzer, T., Kruszka, P., Muenke, M., 2015. Executive Function and Adaptive Behavior in Muenke Syndrome. J. Pediatr. 167, 428-434. https://doi.org/10.1016/j.jpeds.2015.04.080
Zhou, S., Xie, Y., Tang, J., Huang, J., Huang, Q., Xu, W., Wang, Z., Luo, F., Wang, Q., Chen, H., Du, X., Shen, Y., Chen, D., Chen, L., 2015. FGFR3 Deficiency Causes Multiple Chondroma-like Lesions by Upregulating Hedgehog Signaling. PLoS Genetics 11, undefined-undefined. https://doi.org/10.1371/journal.pgen.1005214

1. A method of treating a FGFR3-related cognitive deficit in a subject suffering from FGFR3-related skeletal disease in need thereof comprising administering to the subject a therapeutically effective amount of FGFR3 inhibitor.

2. The method according to claim 1 wherein the FGFR3 inhibitor is a tyrosine kinase inhibitor (TKI).

3. The method according to claim 1 wherein the FGFR3 inhibitor is BGJ398.

4. The method according to claim 1 wherein the FGFR3-related skeletal disease is hypochondroplasia (HCH), achondroplasia (ACH), thanatophoric dysplasia (TD), craniosynostosis or dwarfism.

5. The method according to claim 4 wherein the FGFR3-related skeletal disease is hypochondroplasia (HCH).

6. The method according to claim 4 wherein the FGFR3-related skeletal disease is achondroplasia (ACH).

7. The method according to claim 4 wherein the FGFR3-related skeletal disease is craniosynostosis.

8. The method according to claim 7 wherein the craniosynostosis is Crouzon syndrome with acanthosis nigricans (CAN).

9. The method according to claim 1 wherein the FGFR3-related skeletal disease are is caused by expression in the subject of a constitutively active FGFR3 receptor mutant.

10. The method according to claim 9 wherein the constitutively active FGFR3 receptor mutant is a N540K, K650N, K650Q, M528I, I538V, N540S or N540T mutant.

11. The method according to claim 9 wherein the constitutively active FGFR3 mutant is a A391E mutant.


SEQUENCE LISTING (3 sequences)

SEQ ID NO: 1 - protein, 806 aa, Homo sapiens
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT

SEQ ID NO: 2 - DNA, 19 bp, Artificial Sequence
  Synthetic Primer
gtgggggttctgcggttgg

SEQ ID NO: 3 - DNA, 20 bp, Artificial Sequence
  Synthetic Primer
tgacaggcttggcagtacgg